(19) United States
(12) Patent Application Publication
(10) Pub. No.: US 2026/0137644 A1
(43) Pub. Date: May 21, 2026

(54) COMPOSITIONS INCLUDING CAFFEIC ACID PHENETHYL ESTER

(71) Applicant: XMK THERAPEUTICS INC., Toronto (CA)

(72) Inventors: Kenneth YIP, Toronto, Ontario (CA); Xiao ZHAO, Houston, TX (US)

(73) Assignee: XMK THERAPEUTICS INC., Toronto, ON (CA)

(21) Appl. No.: 19/128,270

(22) PCT Filed: Oct. 10, 2023
(86) PCT No.: PCT/US2023/034820

§ 371 Date: May 8, 2025

Related U.S. Application Data
(60) Provisional application No. 63/382,928, filed on Nov. 9, 2022.

(51) Int. Cl. A61K 31/216; A61K 9/00; A61K 9/06; A61K 31/045; A61K 31/05; A61K 31/07; A61K 31/145; A61K 31/165; A61K 31/167; A61K 31/19; A61K 31/192; A61K 31/197; A61K 31/20; A61K 31/203; A61K 31/255; A61K 31/327; A61K 31/351; A61K 31/353; A61K 31/355; A61K 31/4166; A61K 31/4436; A61K 31/455; A61K 31/585; A61K 31/60; A61K 31/65; A61K 31/7034; A61K 31/7048; A61K 31/7056; A61K 31/728; A61K 33/30; A61K 36/23; A61K 36/53; A61K 36/61; A61K 36/738; A61K 36/886; A61K 36/8962; A61K 45/06; A61P 17/02; A61P 17/10

(52) U.S. Cl. CPC A61K 31/216; A61K 9/0014; A61K 9/06; A61K 31/045; A61K 31/05; A61K 31/07; A61K 31/145; A61K 31/165; A61K 31/167; A61K 31/19; A61K 31/192; A61K 31/197; A61K 31/20; A61K 31/203; A61K 31/255; A61K 31/327; A61K 31/351; A61K 31/353; A61K 31/355; A61K 31/4166; A61K 31/4436; A61K 31/455; A61K 31/585; A61K 31/60; A61K 31/65; A61K 31/7034; A61K 31/7048; A61K 31/7056; A61K 31/728; A61K 33/30; A61K 36/23; A61K 36/53; A61K 36/61; A61K 36/738; A61K 36/886; A61K 36/8962; A61K 45/06; A61P 17/02; A61P 17/10

(57) ABSTRACT

A method of treating acne, of treating UV or radiation induced injury, of reducing the appearance of scars, and/or of treating mucositis, including topically applying a composition including CAPE to a human or other mammal in need thereof, in a therapeutically effective amount and/or from about 1% to about 50% CAPE by weight.

### FIELD OF THE INVENTION

The present invention relates to compositions and methods for the treatment of acne, UV or radiation therapy induced injury, preventing scars and/or reducing the appearance of scars, and/or treatment of mucositis.

### BACKGROUND OF THE INVENTION

### Acne

Acne vulgaris, also known as common acne, is a common disease of the skin which can cause an undesirable appearance on the face, back, and other areas of the body. More than 50 million Americans experience some form of acne. Acne makes up approximately 20% of the visits to dermatologists, and affects approximately 80% of young adults and adolescents.

The management of acne is challenging, especially considering the chronicity of the disease and the variability in response to treatments. Acne management can be complex, because the disease is multifactorial, involving various etiological features, including follicular hyperkeratinisation, increased sebum production, P. acnes proliferation, inflammation and hyperkeratinization.

The skin comprises sebaceous glands which secrete sebum, an oily substance which is a mixture of fat and the debris of dead cells. These cells are constantly replaced by new growth at the base of the sebaceous glands. In humans, sebaceous glands are primarily associated with hair follicles but also occur in hairless areas of the skin.

While sebum serves the purpose of lubricating and protecting the hair and skin and preventing drying and irritation of membranes, it may also be detrimental if it is blocked from exiting the follicle or duct. In acne vulgaris, desquamated (shed) follicular cells and sebum may form a plug which blocks the excretion of sebum from the follicle. This manifests as a “whitehead” also called a closed comedone. Though different theories have been proposed for the dark color of another type of skin blemish commonly known as a “blackhead,” it has been reported that oxidized sebum oil (perhaps mixed with other substances such as melanin) may be responsible. This oxidized oil may harden in the follicle, contributing to the plugging of the normal sebum drainage of the follicle.

Cutibacterium acnes (formerly Propionibacterium acnes) is an anaerobic bacterium that has been reported to be a primary pathogenic agent involved with the development of inflammatory acne and comedogenesis. The plugged follicle provides a favorable anaerobic environment complete with a nourishment source (sebum) for the organism. Overgrowth of C. acnes has been reported to cause destruction of the lining of the follicle, which allows follicular material to enter the dermis.

Specifically, C. acnes hydrolyzes the oil in the sebum, causing the release of free fatty acids. This release of free fatty acids into the surrounding tissue due to rupture of the sebaceous gland causes inflammation. Certain terminology has been generally ascribed to skin blemishes resulting from inflammation, while recognizing that categorization may be subjective to a certain extent. A “pustule” is terminology ascribed to a skin blemish resulting from inflammation very near the surface of the skin. A “pimple” is terminology ascribed to a skin blemish resulting from deeper inflammation. A “cyst” is terminology ascribed to a skin blemish resulting from still deeper inflammation. All of these terms are inclusive in the term acne as herein.

Other bacterial species beyond C. acnes may also contribute to acne. Specifically, it has been suggested that acne may be caused by an imbalance of the different species of bacteria normally populating the skin. While C. acnes is considered a key player, it is highly abundant in the pores of individuals both with and without acne. Other bacterial species that may be implicated in acne are Staphylococcus strains, including Staphylococcus epidermidis and Staphylococcus aureus.

Systemic therapy for acne usually involves prescription antibiotics, such as erythromycin, tetracyclines, and clindamycin, however in recent times physicians have become reluctant to over-prescribe antibiotics because resistance may be developed by not only acne-causing bacteria but other bacteria which are the causative agents of other more serious diseases. Furthermore, systemic administration may cause undesired side effects, as relatively high levels of the drug must circulate throughout the entire body.

Topical antibiotics which have been utilized to attempt to inhibit the overgrowth of Cutibacterium acnes are clindamycin, erythromycin, tetracycline, and metronidazole. Each of these topical antibiotics reportedly cause side effects and widespread use also contributes to the risk of bacterial resistance.

Moreover, current treatments for acne fail to produce satisfactory results. Prior treatments often take away some inflammation or visible symptoms, or remove some of the infected sebum or discharge thereof, but do not cure or eliminate the disorder. Further, patients on such treatment may achieve only some improvement in their condition over long periods of several weeks or months. Therefore, there exists an urgent need for new therapeutic treatments that reduce systemic side effects and also minimize antibiotic resistance.

### UV or Radiation Induced Cutaneous Injury

Human skin can be injured by ultraviolet (UV) radiation, including solar rays, radiation from tanning beds, radiation to treat skin cancer. For example, overexposure to solar radiation can cause simple sunburn or erythema, along with burns of varying severity.

Additional unwanted effects of overexposure to radiation include tanning, age spots, immune system suppression, photosensitivity and photo allergies. Sun exposure can cause the skin to lose elasticity and form wrinkles, yielding prematurely aged skin. Primary symptoms include erythema, warmth, tenderness, edema, and blistering.

Ultraviolet radiation typically refers to the part of electromagnetic spectrum of less than 400 nm. It has been divided into three main components: the UVA (320 to 400 nm), the UVB (290 to 320 nm), and the UVC (200 to 290 nm).

In terms of solar radiation, the UVC is completely absorbed by stratospheric ozone and does not reach the earth's surface. UVB radiation is 1000 times more potent at inducing erythema than UVA radiation and is a principal cause of natural sunburn. UVA comprises the majority of UV radiation reaching the Earth's surface (about 90% at midday) and therefore also accounts for a significant percentage of the acute and chronic cutaneous effects of UV radiation.

Further, since the advent of tanning salons utilizing ultraviolet wavelengths in the UVA-spectrum, UVA-induced sunburn is becoming more prevalent.

Moreover, injury to the skin and underlying tissues from acute exposure to a large external dose of radiation is referred to as cutaneous radiation injury (CRI). This is especially true with acute exposures to beta radiation or low-energy X-rays, because beta radiation and low-energy X-rays are less penetrating and less likely to damage internal organs than gamma radiation is. CRI can occur with radiation doses as low as 2 Gray (Gy) or 200 rads and the severity of CRI symptoms will increase with increasing doses. Many cases arise from a frequently occurring complication of radiation therapy. In fact, it has been noted that nearly 90% of patients having received radiation therapy underwent moderate to skin reactions, which could reduce quality of life and adversely affect disease treatment.

Some cases of radiation cutaneous injury have also occurred when people inadvertently came in contact with unsecured radiation sources from food irradiators, radiotherapy equipment, or well depth gauges. In addition, cases of CRI have occurred in people who were overexposed to x-radiation from fluoroscopy units.

Early signs and symptoms of CRI are itching, tingling, or a transient erythema or edema without a history of exposure to heat or caustic chemicals. Exposure to radiation can damage the basal cell layer of the skin and result in inflammation, erythema, and dry or moist desquamation. In addition, radiation damage to hair follicles can cause epilation. Transient and inconsistent erythema (associated with itching) can occur within a few hours of exposure and be followed by a latent, symptom-free phase lasting from a few days to several weeks. After the latent phase, intense reddening, blistering, and ulceration of the irradiated site are visible. Depending on the radiation dose, a third and even fourth wave of erythema are possible over the ensuing months or possibly years.

The precise biochemical pathways that lead to UV radiation induced cutaneous injury are not well understood, but appear to involve multiple inflammatory mediators, including histamine, prostaglandins and cytokines. Histamine levels are elevated in suction blister fluid obtained during UVB and UVA induced erythema in humans, returning to normal after 24 hours although erythema is still prominent. Prostaglandin E2 increased to approximately 150 percent of control levels after 24 hours and then diminished. Prostaglandins are known to evoke redness and pain in human skin after intradermal injection, and their presence in elevated amounts in suction blisters after UV irradiation suggests their involvement in sunburn erythema. UV irradiation is also a potent stimulus for keratinocyte release of cytokines with local and systemic proinflammatory and immunomodulatory actions.

Current treatment for sunburn includes the systemic administration of aspirin or nonsteroidal anti-inflammatory drugs (NSAIDs) to inhibit the cyclooxygenase pathway and thereby reduce prostaglandin production. NSAIDs work best if administered within the first several hours after exposure. Systemic corticosteroids are often employed and like shorten the course of the pain accompanying severe sunburn. But corticosteroids should not be given to patients with severe burns because they increase the risk of infection. Topical steroids have shown minimal, if any benefit.

Home topical remedies include taking a cool bath with oatmeal or baking soda; and spreading the juice of a cut potato, lavender essential oil, or chamomile on the burn.

While these topical remedies may help soothe the skin or temporarily relieve the pain associated with sunburn, they are not a treatment for the underlying inflammation that defines sunburn. Moreover, patients with allergies, sensitive stomachs, or potential negative drug interactions may not be able to tolerate systemic NSAID treatment. Thus, a topical treatment that effectively reduces UV and radiation induced cutaneous injury is urgently needed.

### Scars

Scars are areas of fibrous tissue replacing normal skin or other tissue after injury or disease, as an expression of the natural healing process of the injured skin or tissue. In general, two types of scars seem to be most abundant, in particular hypertrophic and keloid scars. Hypertrophic scars occur usually in areas of thick skin, in the form of raised, red, nodular lesions within the boundaries of the original wound, characterized by hypervascularity, which is responsible for their erythematous appearance. Keloid scars on the other hand mostly have a genetic etiology. The size and shape of keloids have little correlation to the extent of the skin wound, and keloids may grow to form big boney tumors. Other types of frequently occurring scars include contracture scars caused by the loss of a large section of skin following e.g., burns or other injury, where the scars contract or tighten around the wound and that way impact the patient's mobility—and acne scars which may form as a result of severe acne, a skin condition usually caused by hormonal changes.

Wound healing is a complex, tightly regulated process of wound repair, which comprises an inflammation phase, a proliferation phase and a maturation phase. Release of cytokines from the injured tissue cells and wound blood clot initiates the inflammatory phase. The proliferation stage is characterized by cascading coagulation wherein an increasing number of fibroblasts with increasing density is released for the synthesis of extracellular matrix and collagen. As soon as sufficient provisional matrix is generated, the maturation stage is initiated. The maturation stage is characterized by a shift in balance from scar remodeling towards scar degradation and is accompanied by extracellular matrix reorganization and reduction. The pathophysiologic mechanisms leading to keloid or hypertrophic scar formation are still unknown, although many different theories have been proposed. Position of the injury and ethnic background may predispose a patient to the development of keloid or hypertrophic scars, besides other parameters.

Every year, millions of injured patients become affected by keloid and hypertrophic scar formation. It is estimated that about 80% of the population has one or more scars. The incidence of hypertrophic scars following surgery is said to be about 40-70%, whereas it is higher (up to 91%) following burn injury. Although hypertrophic and keloid scars may develop at any age, patients between 10 and 30 years old seem to be most often affected. It may be true that the presence of scars is not life-threatening and affects the functioning of the human body in a small number of cases only, nevertheless their psychological impact on the human well-being may not be underestimated.

A wide variety of treatment methods and agents has been developed, which aim at masking or covering scars or reducing their visibility. Unfortunately, none of the techniques developed to date permits achieving permanent scar removal. Surgical excision of the scar in combination with steroids and/or silicone treatment are associated with frequent scar recurrence. Scars older than 6-12 months seem to respond poorly to pressure therapy, the pressure dressing is moreover sensed as uncomfortable by the patient. Other treatments include radiation therapy, laser therapy, cryotherapy, intralesional administration of corticosteroids and topical application of steroid creams. Cosmetic creams and ointments based on gel technology which aim at hydrating and/or masking scars, provide a limited physical barrier only when applied to scar tissue. As a result, their effect in scar healing and reduction is limited as well.

More recently developed methods aiming at improving the signs and symptoms of scars and at preventing the development of abnormal scarring, include topical silicone therapy in particular topical application of silicone gels and silicone gel sheets. Silicones may soften, smoothen and flatten scar tissue, they may assist in reducing itching and light pain, they are suitable for use with both keloid and hypertrophic scars. Silicones are appreciated for their humidifying properties and their ability to act as a carrier for other active ingredients e.g., UV-protectors, anti-microbial agents and humectants. The use of silicone gel is well accepted by patients because the sheet formed by it is nearly invisible sheet and dries fairly quickly when applied correctly in a thin layer. The use of silicone sheets poses several limitations, in particular because application to certain body parts and to large areas or near joints as well as use on the face or other areas where the contours or motility of the skin make it difficult to ensure adequate contact and coverage, may be difficult. Silicone sheets must be washed carefully and sufficiently often to prevent infection. A major downside of silicone gel sheeting is the fact that the sheet adheres to the skin. Frequent removal may cause removal of varying amounts of superficial stratum corneum and skin stripping in the normal skin adjacent to the wound, leading to changes in the skin barrier function, in particular increased transepidermal water loss. An inflammatory skin reaction may develop in the skin adjacent to the wound.

Therefore, it is clear that new compositions and method for preventing scars, as well as treating and reducing the appearance of scars, are urgently needed.

### Mucositis

Mucositis is the painful inflammation and ulceration of the mucous membranes lining the digestive tract, usually as an adverse effect of chemotherapy and radiotherapy treatment for cancer. Ridge, et al. “Head and Neck Tumors” in Pazdur R, Wagman LD, Camphausen KA, Hoskins W J (Eds) Cancer Management: A Multidisciplinary Approach. [11 Ed.] (2008). It can have many origins but, for example, often arises as a side effect of chemotherapy and radiotherapy for cancer. It can also appear in patients who are otherwise immunosuppressed or for idiopathic reasons. Mucositis can occur anywhere along the gastrointestinal (GI) tract. Oral mucositis is marked by inflammation and ulceration in the mouth and is a common and often debilitating complication of cancer treatment. Sonis (2004) J Supportive Oncology 2(Suppl 3): 3-8. Both oral and gastrointestinal mucositis may result from radiotherapy and chemotherapy. Both forms of mucositis have a significant impact on health, quality of life and economic outcomes that are associated with treatment.

Historically, chemotherapy induced mucositis has been conceptualized as resulting from cytotoxic damage to rapidly dividing submucosal basal cells, which results in epithelial cell damage, whereas radiation-induced mucositis results from damage to the epithelial cells exposed to radiation.

Interestingly, the primary damage response according to a proposed Five-Stage Model includes the activation of p53 and NF-KB, the latter two being the most significant events in propagating the damage response. Thus, generalized inflammatory pathways that have been implicated in CAPE bioactivity play a likely role in mucositis disease and disease response.

One ultimate result of NF-KB activation is the production of inflammatory cytokines including IL-1B and IL-6, which directly lead to tissue damage and cell death. While during this stage, mucositis may still be subclinical, it may quickly lead to ulceration and visually apparent lesions in the mucosa: at this stage, a very high risk of bacterial colonization emerges, including the development of sepsis.

Moreover, newer, molecularly targeted drugs have also been noted as having a side effect of mucositis. So-called “targeted-agent” mucositis is frequently referred to as stomatitis to distinguish it from generalized, non-specific chemotherapy or radiotherapy induced mucositis. The mechanism is not fully understood but may involve poor healing following repeated chemico-microtrauma. Specific targeted agents associated with mucositis include anti-angiogenic tyrosine kinase inhibitors (TKIs), including sunitinib, sorafenib, axitinib, pazopanib, and cabozantinib.

Thus, it is exceedingly evident that only recently has the complex pathogenesis of mucositis been appreciated, including the realization that mucositis reflects the dynamic interactions of all of the cells and tissue types that comprise the epithelium and submucosa. The identification of the molecular events that lead to treatment-induced mucosal injury has provided targets for mechanistically based interventions to prevent and treat mucositis.

The most common symptoms of mucositis include redness, dryness, or swelling of the mouth, burning or discomfort when eating or drinking, open sores in the mouth and throat, abdominal cramps, and tenderness or rectal redness or ulcers. Essentially mucositis involves the inflammation of the lining of the mouth and digestive tract, and frequently occurs in cancer patients after chemotherapy and radiation therapy. The cheek, gums, soft plate, oropharynx, top and sides of tongue, and floor of the mouth may be affected, as well as the esophagus and rectal areas. Along with redness and swelling, patients typically experience a strong, burning pain.

Although there are factors that increase the likelihood and severity of mucositis, there is no reliable manner to predict who will be affected. Not only is mucositis more common in elderly patients, but the degree of breakdown is also often more debilitating. The severity of mucositis tends to be increased if a patient exercises poor oral hygiene or has a compromised nutritional status. A preexisting infection or irritation to the mucous membrane may also result in a more severe case of mucositis.

Currently there is no real cure for mucositis, treatment is aimed at prevention and management of symptoms. However, some “swish and spit” treatments for mucositis do exist. For example, morphine and oral ketamine are both used as swish and spit formulations that have been used to treat the painful symptoms associated with mucositis. However, these treatments are used primarily for pain management and do not provide any treatment of the underlying condition. Dexamethasone is also used, although it comes with several concomitant side effects such as bone loss, bruising, and pregnancy risks.

Therefore, a need exists for treatments that reduce the painful symptoms and cell damage arising from oral and GI mucositis and that are also safe and effective.

### SUMMARY OF THE INVENTION

In one embodiment herein is a method of treating acne, comprising: topically applying a composition comprising CAPE to a subject in need thereof in a therapeutically effective amount and/or comprising from about 1% to about 50% CAPE by weight.

In another embodiment herein is a method of treating UV or other radiation induced injury, comprising: topically applying a composition comprising CAPE to a subject in need thereof in a therapeutically effective amount and/or comprising from about 1% to about 50% CAPE by weight.

In another embodiment herein is a method of preventing and/or reducing the appearance of scars, comprising: topically applying a composition comprising CAPE to a subject in need thereof in a therapeutically effective amount and/or comprising from about 1% to about 50% CAPE by weight.

In another embodiment herein is a method of treating mucositis, comprising: topically applying a composition comprising CAPE to a subject in need thereof in a therapeutically effective amount and/or comprising from about 1% to about 50% CAPE by weight.

In another embodiment the composition further comprises a pharmaceutically acceptable base in the form of a liquid, aqueous, or semi-solid composition.

In another embodiment herein is that the pharmaceutically acceptable base is a water and oil emulsion (such as O/W, W/O, W/O/W), dispersion, suspension, colloid, liposome, microemulsion, nanoemulsion, clear solution, or anhydrous composition.

In another embodiment herein is that the pharmaceutically acceptable base comprises a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, or skin surface implant.

In another embodiment herein is that the pharmaceutically acceptable base further comprises at least one of the following skin-beneficial ingredients: skin cleansers, surfactants which may be cationic, anionic, non-ionic, amphoteric or zwitterionic, skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In another embodiment herein is that the pharmaceutically acceptable base further comprises at least one of the following solubilizing-penetration enhancing agents: alcohols, hydroxy acid esters, or glycols.

In another embodiment herein is that the composition further comprises an antibiotic, for instance, the antibiotic comprises erythromycin, clindamycin, minocycline, doxycycline, and further optionally comprising benzoyl peroxide.

In another embodiment herein is that the antibiotic is active against Cutibacterium acnes, Staphylococcus epidermidis, or Staphylococcus aureus.

In another embodiment herein is that the compositions further comprises an anti-inflammatory agent.

In another embodiment herein is that the anti-inflammatory agent comprises clindamycin, erythromycin, dapsone, aloe vera or tea tree oil.

In another embodiment herein is that the composition further comprises one or more anti-scarring agents.

In another embodiment herein is that the anti-scarring agent comprises glycolic acid, kojic acid soap, niacinamide, silicone sheeting, silicone gel, mediderm gel, Vitamin E oil, Derma E scar gel, hydrocolloid formulation, phenols, catechins, epigallocatechin gallate, rosehip oil, allicin, allantoin, onion extract, arbutin glycoside, hyaluronate or thyme oil.

In another embodiment herein is that the composition further comprises at least one analgesic agent.

In another embodiment herein is that the analgesic agent comprises aloe vera, lidocaine, capsaicin, menthol, or a nonsteroidal anti-inflammatory drug (NSAID).

In another embodiment herein is that the composition further comprises at least one emollient, e.g., the emollient comprises a fatty ester, a fatty alcohol, petrolatum, dimethicone, and combinations thereof.

In another embodiment herein is the composition further comprises at least one emulsifier.

In another embodiment herein is that the composition further comprises at least one essential oil.

In another embodiment herein is that the composition comprising CAPE further comprises a humectant.

In another embodiment herein is that the humectant comprises aloe vera, cocoa butter, Vitamin E, honey, or a combination thereof.

In another embodiment herein is that the applying topically is conducted monthly, weekly, daily, or up to six times per day.

In another embodiment herein is that the subject is an animal.

In another embodiment herein is that the animal is a mammal.

In another embodiment herein is that the composition does not contain antepilin C, quercetin, chloredan diterpene, P-coumaric acid and/or cinnamic acid.

In another embodiment herein is that the composition further comprises at least one of the following anti-acne agents: salicylic acid or derivative thereof, ester of a salicylic acid or derivative thereof, azelaic acid, Dapsone, benzoyl peroxide, zinc salts including zinc pyrithione and zinc sulfate, resorcinol, Vitamin A, Vitamin E, Vitamin B5, polyhydroxy acids (PHAs), alpha hydroxy acids (AHAs), beta hydroxy acids (BHAs), niacinamide, centella asiatica, cortisone, cortisone injection, hydrocortisone, hyaluronic acid, oligopeptides, hydrocolloid formulations, tea tree oil or retinol.

In another embodiment herein is that the composition further comprises a prescription anti-acne drug.

In another embodiment herein is that the prescription anti-acne drug comprises isotretinoin, tretinoin, adapalene, tazarotene, spironolactone, or combination birth control.

In another embodiment herein is that the methods described herein above further comprise(s) topically applying the composition comprising CAPE to a patient previously undergoing a treatment regimen to treat acne.

In another embodiment herein is that the treatment regimen comprises: isotretinoin, salicylic acid, benzoyl peroxide, zinc salts including zinc pyrithione and zinc sulfate, resorcinol, Vitamin A, or Vitamin E.

In another embodiment herein is that the composition further comprises sulfur spot treatment and further optionally comprises resorcinol and/or sodium sulfacetamide.

In another embodiment herein is that a method of treating acne, comprising: injecting a composition comprising CAPE to a subject in need thereof in a therapeutically effective amount and/or comprising from about 0.1 mg/kg to about 10 mg/kg.

In another embodiment herein is that the injecting is administered locally at the site of the acne.

In another embodiment herein is that the injecting is intravascular or intraperitoneal.

In another embodiment herein is a method of treating heat rash, comprising: topically applying a composition comprising CAPE to a subject in need thereof in a therapeutically effective amount and/or comprising from about 1% to about 50% CAPE by weight.

In another embodiment herein is that method of treating UV or radiation therapy induced injury, comprising: locally injecting a composition comprising CAPE at a site of the injury to a subject in need thereof in a therapeutically effective amount and/or comprising from about 0.1 mg/kg to about 10 mg/kg.

In another embodiment herein is that method of treating UV or radiation therapy induced injury, comprising: orally administering a composition comprising CAPE to a subject in need thereof in a therapeutically effective amount and/or comprising from about 10 mg to about 1000 mg.

In another embodiment herein is that methods described herein above can also include topically applying a composition comprising CAPE in conjunction with Mederma Advanced Scar Gel™, SkinCeuticals Phyto+Botanical Gel™, Cica-Care Gel Sheet™, Cimeosil Scar and Laser Gel™, ScarAway Silicone Scar Gel™, Tosowoong Green Tea Natural Pure Essence™, Honeydew Scar Cream™, Mederma for Kids™, Derma E Scar Gel™, Rejuran Advanced Scar Cream™, Cera Ve Retinol Serum™, Musely the Spot Cream™, Differin Resurfacing Scar Gel™, SkinMedica Scar Recovery Gel™, DermalMD Scar Treatment™, or a combination thereof.

In another embodiment herein is a method of treating mucositis, comprising: orally administering a liquid composition comprising CAPE to an oral mucosa of a subject in need thereof in a therapeutically effective amount and/or comprising from about 1% to about 50% CAPE by weight.

In another embodiment herein is that the subject is currently undergoing cryotherapy or ice chip therapy to treat mucositis, wherein the subject is optionally receiving infusion of fluoroacil treatment.

In another embodiment herein is that the liquid composition comprising CAPE is administered with a mucoadhesive hydrogel rinse and/or a calcium phosphate rinse.

In another embodiment herein is A method of prophylactically preventing mucositis, comprising: orally administering a liquid composition comprising CAPE to an oral mucosa of a subject undergoing head and neck cancer radiotherapy, wherein the administering occurs in a therapeutically effective amount and/or comprising from about 1% to about 50% CAPE by weight.

In another embodiment herein is that the liquid composition comprising CAPE is administered before chemotherapy or radiotherapy to treat cancer to decrease the incidence of mucositis.

In another embodiment herein is that the liquid composition comprising CAPE is administered in conjunction with a benzydamine mouthwash.

In another embodiment herein is that the liquid composition comprising CAPE is administered in conjunction with an opiate mouthwash.

In another embodiment herein is that the opiate mouthwash comprises morphine, fentanyl, or codeine.

In another embodiment herein is that the liquid composition comprising CAPE is administered in conjunction with a “salt-and-soda” mouthwash.

In another embodiment herein is that the salt-and-soda mouthwash comprises a normal saline solution or a sodium bicarbonate solution.

In another embodiment herein is that the liquid composition comprising CAPE further comprises an anesthetic, an antihistamine, an antibiotic, or a steroid.

In another embodiment herein is that the anesthetic comprises lidocaine.

In another embodiment herein is that the steroid comprises dexamethasone.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows the results of survey regarding a reduction of pimple size after using an acne spot cream comprising CAPE.

FIG. 2 shows the results of a survey regarding a reduction of pimple redness after using an acne spot cream comprising CAPE.

FIG. 3 shows the results of survey regarding a reduction of a number of pimples after using an acne spot cream comprising CAPE.

FIG. 4 shows the results of survey regarding a level of embarrassment before and after using an acne spot cream comprising CAPE.

FIG. 5 shows the results of survey regarding a level of self-consciousness before and after using an acne spot cream comprising CAPE.

FIG. 6 shows the results of survey regarding a level of upsetness before and using an acne spot cream comprising CAPE.

FIG. 7 shows the results of survey regarding a difference made to participants' pimples after using an acne spot cream comprising CAPE.

FIG. 8 shows a time course of with or without CAPE treatment in an acute UV injury.

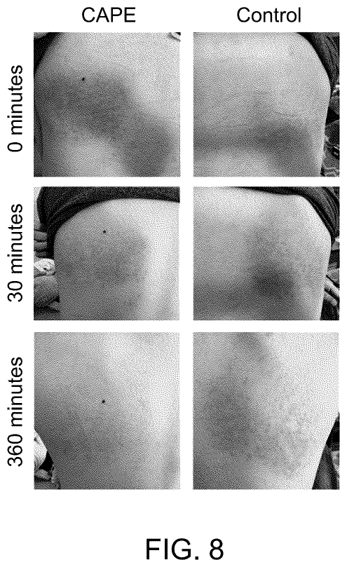

### DETAILED DESCRIPTION OF THE PREFERRED EMBODIMENTS

Caffeic acid phenethyl ester (2-phenylethyl(2E)-3-(3,4-dihydroxyphenyl)acrylate or 2-phenylethyl (E)-3-(3,4-dihydroxyphenyl)prop-2-enoate or 2-phenylethyl 3-(3,4-dihydroxyphenyl)prop-2-enoate, or CAPE) is a bioactive phenolic compound found in honeybee hive propolis. Propolis is a resinous mixture produced by honeybees by mixing saliva and beeswax with exudate gathered from tree buds, sap flow, or other botanical sources, and used as a sealant in the hives. Propolis has long been believed to have beneficial effects on, for example, inflammation and chronic diseases, and, for example, was widely used for its medicinal properties, including wound healing, by the ancient Egyptians.

Importantly, bee propolis is a mixture containing hundreds of polyphenolic compounds, of which CAPE is one.

The present inventors have realized that compositions comprising CAPE may lead to significant therapeutic improvements for a variety of skin disorders. For example, CAPE may lead to an improvement in acne symptoms. CAPE may also reduce the symptoms of UV radiation induced cutaneous injury, such as by sunburn. CAPE may also reduce the symptoms of non-UV radiation induced cutaneous injury, such as may be incurred by radiation treatment. Furthermore, CAPE may also prevent or reduce the appearance of scar tissue. Finally, CAPE may constitute a beneficial treatment for mucositis.

CAPE is known to have both anti-microbial and anti-inflammatory properties.

CAPE's anti-inflammatory activity may involve the inhibition of arachidonic acid release from the cell membrane; it, in return, inhibits the COX-1 and COX-2 activity as well as suppresses the activation of gene responsible for COX-2 expression [22-25]. In carrageenin-induced inflammation, CAPE suppresses both exudate volume and leukocytes relocation.

CAPE's anti-inflammatory properties may also originate from its mechanism as an inhibitor of the nuclear factor kB (NF-KB). NF-KB is broadly implicated in human inflammation pathways. CAPE has also been implicated in mediating the inhibition of initial and late steps in T-cell receptor-mediated T-cell activation, which is a possible mechanistic basis for the immunomodulatory and anti-inflammatory activities of CAPE. CAPE inhibition of both interleukin-2 (IL-2) gene transcription and synthesis has also been observed. In CAPE-treated Jurkat cells, there was restricted binding of NF-KB to DNA and transcriptional activity of a Gal4-p65 hybrid protein. In addition, CAPE-mediated inhibition of binding with DNA and transcriptional activity of NFAT in CAPE-treated Jurkat cells was also seen.

CAPE has also been documented as having potent antimicrobial activity. For example, CAPE has been shown to active against Enterococcus faecalis, Listeria monocytogenes, Staphylococcus aureus, and Haemophilus influenzae.It has been hypothesized that bacterial RNA, DNA, and cellular proteins are possible targets of CAPE. Moreover, poly(lactic-co-glycolic acid) (PLGA) sutures containing CAPE were proposed to have antibacterial activity against Staphylococcus aureus and Escherichia DH5 α bacteria, wherein the antimicrobial activity of CAPE was attributed to the synthesis of reactive oxygen species (ROS) that destroy the outer bacterial membrane.

Without being bound by theory, it is possible that the unique combination of anti-microbial and anti-inflammatory properties of CAPE may contribute to its effectiveness in treating acne, UV and other radiation induced cutaneous injury, and prevention and/or reduction in the appearance of scars.

With respect to acne, it is possible that CAPE may have antimicrobial activity against the particular bacterial species that are causative of acne. It has also been noted that some bacterial species implicated in acne exist in people who do not experience acne, suggesting that acne may arise, at least partially, from a general imbalance of the broad plethora of species inhabiting healthy skin. Thus, and without being bound by theory, CAPE may have bioactivity against any number of species that relate to an overall bacterial imbalance of the skin that may contribute to acne. Without being bound by theory, it is also possible that the unique combination of antibacterial, anti-inflammatory and other biological activity of CAPE may contribute to an effectiveness in treating acne.

With respect to UV radiation injury, such as sunburn, or other radiation induced injury, and without being bound by theory, it is possible that that CAPE's broad anti-inflammatory activity, in addition to synergistic antibacterial activity, may reduce symptoms of such cutaneous injury, whether from natural solar radiation, tanning beds, or exposure to therapeutic radiation therapy or other sources of man-made radiation.

With respect to an effectiveness of CAPE in preventing and/or reducing the appearance of scars, it is believed that these bioactive properties of CAPE may also prevent and/or reduce the appearance of scar tissue when administered as a therapeutic formulation.

With respect to mucositis, recent studies have identified activation of key inflammatory pathways, including NF-KB and IL-6, as constituting primary damage response mechanisms of the human body to chemotherapy or radiotherapy as applied to mucosal membranes. These initial response mechanisms are generated by, as has been hypothesized, repeated microtrauma to epithelial cells of the mucosa as caused by chemotherapy or radiotherapy. Moreover, subsequent downstream effects of microbial attack of the damaged cells can lead to widespread cell damage, death, and even sepsis. Thus, it can be seen that the anti-inflammatory and antimicrobial effects of CAPE may play a role in either preventing or reducing the severity of mucositis.

As mentioned above, the present inventors have found that compositions comprising CAPE may be effective in treating acne, UV or other radiation induced injury, including sunburn, in preventing and/or reducing the appearance of scars, and in treating mucositis. As used herein, the terms “treating” and “treatment” refer to reduction in severity and/or frequency of symptoms, elimination of symptoms and/or underlying cause, prevention of the occurrence of symptoms and/or their underlying cause, and improvement or remediation of damage. The present method and formulations of “treating” a patient, as the term is used herein, thus encompasses both prevention in a predisposed individual and treatment in a clinically symptomatic individual.

In some embodiments, a method of treatment may comprise topically applying a composition comprising CAPE to a subject in need thereof in a therapeutically effective amount. In the composition, the percentage of CAPE may be from about 1% to about 50% by weight.

As used herein, a subject “in need thereof” refers to a subject currently suffering from or predisposed in the future to suffer from acne, UV or radiation induced cutaneous injury, or scars.

In some embodiments, the composition comprising CAPE may consist essentially of CAPE. In other embodiments, the composition may further comprise at least one other active agent, as discussed in detail below.

In some embodiments, the composition comprising CAPE may further comprise an antibiotic. As used herein, the term “antibiotic” refers to an agent that either kills or inhibits the growth of a microorganism (e.g., virus, fungus, bacteria, protozoans, etc.). Without being bound by theory, it is possible that the combination of CAPE and one or more antibiotics may have a beneficial therapeutic effect, increasing effectiveness while reducing systemic side effects and/or the likelihood of antibiotic resistance. Non-limiting examples of antibiotics that may be administered in conjunction with CAPE include erythromycin, clindamycin, minocycline, and doxycycline.

In other preferred embodiments, the composition will include other antimicrobial agents that are active against bacteria that can populate the skin, including but not limited to C. acnes, S. epidermidis, and S. aureus and Propionibacterium strains including Propionibacterium avidum and Propionibacterium granulosum.

In other embodiments, the composition comprising CAPE further comprises one or more anti-inflammatory agents. A wide variety of anti-inflammatory agents can be envisioned, and will be clear to the person skilled in the art, but some may include clindamycin, erythromycin, dapsone, aloe vera, tea tree oil, and combinations thereof.

Other anti-inflammatory agents that may be present in some embodiments including non steroidal anti-inflammatory drugs (NSAIDs). As used herein, the term “nonsteroidal anti-inflammatory drug (NSAID)” refers to a class of drugs that provides analgesic (painkilling) and antipyretic (fever-reducing) effects, and anti-inflammatory effects. The term “nonsteroidal” distinguishes these drugs from steroids, which, among a broad range of other effects, have a similar eicosanoid-depressing, anti-inflammatory action. As analgesics, NSAIDs are unusual in that they are non-narcotic and thus are used as a non-addictive alternative to narcotics. The most prominent members of this group of drugs (aspirin, ibuprofen and naproxen), are all available over the counter in most countries. NSAIDs inhibit the activity of both cyclooxygenase-1 (COX-1) and cyclooxygenase-2 (COX-2), and thereby, the synthesis of prostaglandins and thromboxanes. For the purposes of this application, acetaminophen is also considered an NSAID because it has at least some of its activity through the COX enzymes.

A composition comprising CAPE in some embodiments may also comprise a pharmaceutically acceptable base in the form of a liquid, aqueous, or semi-solid composition. The base is without limitation and is understood to encompass a wide variety of pharmaceutically acceptable bases that are known to the person skilled in the art. For example, the base may comprise a water and oil emulsion, a O/W, W/O, or W/O/W emulsion, a dispersion, suspension, colloid, microemulsion, nanoemulsion, clear solution, or an anhydrous composition.

By “pharmaceutically acceptable,” such as in the recitation of a “pharmaceutically acceptable base,” a “pharmaceutically acceptable carrier,” or a “pharmaceutically acceptable excipient,” it is meant a compound that is not biologically or otherwise undesirable, i.e., the compound may be incorporated into a topical formulation of the invention and administered to a patient without causing any undesirable biological effects or interacting in a deleterious manner with any of the other components of the formulation in which it is contained. A “pharmacologically active” compound refers to an active agent as defined above, or to an analog or derivative thereof having the same type of pharmacological activity as the parent compound.

In some embodiments, additional excipients may be added to the formulation to improve the composition's appeal, such as fragrance, silicone, and humectants, such as glycerin or sorbitol. Excipients may also include those ingredients known in the art to aid in solubilizing, suspending, emulsifying or otherwise stabilizing the active ingredients of the formulation.

In some embodiments, the pharmaceutically acceptable base may comprise a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

Suitable pharmaceutically acceptable carriers, including emulsions such as creams or lotions, ointments, gels, and aqueous solutions suitable for topical application, are disclosed in REMINGTON: THE SCIENCE AND PRACTICE OF PHARMACY 282-291 (Alfonso R. Gennaro ed. 19^{th} ed. 1995), hereby incorporated herein by reference.

In some embodiments, the pharmaceutically acceptable base may further comprise solubilizing-penetration enhancing agents. Without limitation, examples of such agents include alcohols, hydroxy acid esters, glycols, and combinations thereof.

By an “effective” amount or a “therapeutically effective amount” of a pharmacologically active agent is meant a nontoxic but sufficient amount of the drug or agent to provide the desired effect, i.e., prevention or treatment of acne. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

In some embodiments, the composition comprising CAPE will be administered topically to the person in need of acne treatment. The term “topical,” as in a topical administration, is used in its conventional sense to mean delivery of a topical drug or pharmacologically active agent to the skin, as in, for example, the treatment of an inflammatory dermatosis such as acne vulgaris.

The frequency of administration is not particularly limited and may depend on several factors, such as the amount of CAPE or other ingredients in the composition, the type of pharmaceutically acceptable base or carrier (e.g., cream versus solution), the severity of the acne, or any of a number of other possible parameters. For example, the composition comprising CAPE may be administered monthly, weekly, daily, or multiple times per day, such as six times per day or even hourly. Moreover, the composition may be applied topically to the skin and then left for a certain period of time on the skin to maximize contact with the acne, such as for several hours, or overnight. But overall, and notwithstanding anything else in this paragraph, dosages and dosing frequency will be determined by a trained medical professional depending on the activity of the compounds used, the characteristics of the particular topical formulation, and the identity and severity of the acne disorder being treated or prevented. The dosages will preferably be communicated on a label or packaging that accompanies the products of preferred embodiments.

The pH of the topical compositions comprising CAPE are preferably within a physiologically acceptable pH, e.g., within the range of about 5 to about 8, more preferably, of about 5.5 to about 6.5. To stabilize the pH, preferably, an effective amount of a buffer is included. In one embodiment, the buffering agent is present in the aqueous topical formulation in an amount of from about 0.05 to about 1 weight percent of the formulation. Acids or bases can be used to adjust the pH as needed.

The composition comprising CAPE can, in some embodiments of the invention, also comprise pharmaceutically acceptable excipients such as those listed in REMINGTON: THE SCIENCE AND PRACTICE OF PHARMACY 866-885(Alfonso R. Gennaro ed. 19^{th} ed. 1995); and TRANSDERMAL AND TOPICAL DRUG DELIVERY SYSTEMS (Ghosh, T. K. et al. ed. 1997), hereby incorporated herein by reference, including, but not limited to, protectives, adsorbents, demulcents, emollients, preservatives, antioxidants, moisturizers, buffering agents, solubilizing agents, skin-penetration agents, and surfactants. In some embodiments, the topical composition of the invention further includes one or more agents selected from a preservative, a local anesthetic and a skin humectant.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenyl ethyl alcohol, and benzyl alcohol; antibacterial esters, for example, esters of parahydroxybenzoic acid; and other anti-microbial agents such as chlorhexidine, chlorocresol, benzoic acid and polymyxin.

Some embodiments described herein may feature a composition comprising CAPE and further comprising additional skin-beneficial ingredients. These ingredients may include skin cleansers, soaps or surfactants wherein the surfactants may be cationic, anionic, non-ionic, amphoteric or zwitterionic, skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In some embodiments, a surfactant may be utilized in making the composition comprising CAPE. A preferred surfactant is a non-ionic, non-toxic surfactant which is suitable for application to human skin, such as a difunctional block copolymer surfactant. For example, one preferred embodiment is a difunctional block copolymer surfactant which terminates in primary hydroxyl groups. The additional skin-beneficial ingredients may also be fragrances, cosmetic layers including face blender, eye shadow, skin toner, or other make-up component.

In some embodiments, the composition comprising CAPE may further comprise at least one analgesic agent. The type of analgesic agent is not particularly limited and the person skilled in the art will readily recognize the wide range of analgesics available. For example, some embodiments may include the analgesics aloe vera, capsaicin, menthol, an NSAID, and combinations thereof.

In some embodiments, the composition comprising CAPE may include an emollient. An emollient is a moisturizing treatment that covers the skin with a protective film in order to trap in moisture. Examples of emollients that may be present in some embodiments include diisopropyl adipate, oleyl alcohol, lanolin, isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, polyoxypropylene (5) poloxyethylene (20) cetyl ether (PPG-5-Ceteth-20), 2-ethylhexyl isononoate, 2-ethylhexyl stearate, C12 to C16 fatty alcohol, C12 to C16 fatty alcohol lactate, isopropyl lanolate, 2-ethylhexyl salicylate, and mixtures thereof. In some embodiments, the one or more emollients may be a combination of fatty alcohols. In certain embodiments, the one or more emollients may be 1-hexadecanol, acetylated lanolin, behenocyl dimethicone, C12-15 alkyl benzoate, cetearyl octanoate, cocoglycerides, dicaprylate/dicaprate dimethicone copolyol, dimethiconol, dioctyl adipate, glyceryl stearate, isocetyl alcohol, isohexadecane, isopentylcyclohexanone, isopropyl palmitate, lauryl lactate, mineral oil, methoxy peg-22/dodecyl glycol copolymer, myristyl lactate, ocryldodecyl neopentanoate, octyl cocoate, octyl palmitate, octyl stearate, octyldodecyl neopentanoate, polyglyceryl-4 isosterate, polyoxyl 40 stearate, polyoxymethylene urea, potassium sorbate, propylene glycol, propylene glycol isoceth-3 acetate, and propylene glycol myristyl ether acetate.

The emollient may be provided in any suitable amount. For example, in some embodiments, the one or more emollients may be from about 1 to about 50% by weight of the composition comprising CAPE.

In some embodiments, the composition comprising CAPE may also include an emulsifier. Emulsifiers may be nonionic, cationic, anionic, or zwitterionic emulsifiers. Non-limiting examples may include: esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

In some other embodiments, the composition comprising CAPE may also include at least one essential oil. Essential oils are concentrated plant extracts, generally extracted by distillation or mechanical pressing. Some examples of essential oils that may be present in some embodiments include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang.

In some other embodiments, the composition comprising CAPE may be administered in conjunction with a UV protecting agent, more preferably a protecting agent selected from the Group of UV-A and UV-B protecting agents or mixtures thereof. For example, skin affected by acne, UV or other form of radiation, or scar tissue is normally highly sensitive to irradiation by sun-light and in particular to irradiation by UV-light. Due to the incorporation of at least one UV-protecting agent, the risk to further damage and irritation may be reduced. Incorporation of at least one UV-protecting agent further permits minimizing the risk to the formation of irreversible, visible scars on top of the existing scars. Therefore, some embodiments include UV-protecting agents.

The UV-protecting agent is preferably selected from the group of 1-(4-tert.butylfenyl)-3-(4-methoxyfenyl)-propane-1,3-dion, 4-tert-.butyl-4-methoxydibenzoyl methane, 1-phenyl-3-(4-isopropylphenyl)-propane-1,3 dion, 4-methylbenzyllidenecamphor, 2-fenyl-benzimidazole-5-sulfonic acid, octyl methoxycinnamate, octyl salicylate, octycrylene benzophenone-3 homosalate, octocrylate, avobenzone, and menthyl anthranilate, inorganic particulate materials, zinc oxide, silica, iron oxide, titanium dioxide and 2-ethyl-hexyl-p-methoxycinnamate, avobenzone, and mixtures of two or more of these components. Examples of oil soluble UV-A filters include 1-(4-tert.butylphenyl)-3-(4-methoxyfenyl)-propane-1,3-dion, 4-tert-.butyl-4-methoxydibenzoyl methane and 1-fenyl-3-(4-isopropylphenyl)-propane-1,3 dion. Examples of UVB-filters include octylmethoxycinnamate, octycrylene, 4-methylbenzyllidenecamphor, 2-phenyl-benzimidazole-5-sulfonic acid, octyl salicylate, zinc oxide, titanium oxide, iron oxide. Preferably, in the composition of this invention use is made of micronized titanium dioxide which provides UV-A as well as UV-B protection. Penetration of UV radiation impinging the skin or scar tissue (290-370 nm) is blocked because the UV-light dispersing and reflecting properties of the titanium dioxide particles. The titanium dioxide particles may be transparent to light with longer wavelength. More preferably, the composition of this invention comprises a mixture of micronized titanium dioxide and avobenzone (4-tert-.butyl-4-methoxydibenzoyl methane). Avobenzone is an oil soluble UV-A protective agent capable of blocking a large UV-A spectrum (310-400-nm).

In some embodiments, the composition comprising CAPE further comprises a humectant. A humectant is a hydrating material that preserves moisture by providing a protective barrier to the outside air environment and also by having an affinity to hydrogen bonds of water molecules and to skin hydrophilic molecular functionalities. Topical application of cosmetic products containing humectants, (for example, glycerin) can be associated with improvements in barrier function, induction of biomarkers associated with keratinocyte proliferation and wound healing, reduction in melanin intensity, increases in epidermal thickness, and improvements in general skin appearance.

Examples of humectants that may be present in some embodiments include amino acids and derivatives thereof such as proline and arginine aspartate, 1,3-butylene glycol, propylene glycol and water and codium tomentosum extract, collagen amino acids or peptides, creatinine, diglycerol, biosaccharide gum-1, glucamine salts, glucuronic acid salts, glutamic acid salts, polyethylene glycol ethers of glycerine (e. g. glycereth 20), glycerine, glycerol monopropoxylate, glycogen, hexylene glycol, honey, and extracts or derivatives thereof, hydrogenated starch hydrolysates, hydrolyzed mucopolysaccharides, inositol, keratin amino acids, LAREX A-200 (available from Larex), glycosaminoglycans, methoxy PEG 10, methyl gluceth-10 and-20 (both commercially available from Amerchol located in Edison, NJ), methyl glucose, 3-methyl-1,3-butanediol, N-acetyl glucosamine salts, polyethylene glycol and derivatives thereof (such as PEG 15 butanediol, PEG 4, PEG 5 pentaerythitol, PEG 6, PEG 8, PEG 9), pentaerythitol, 1,2 pentanediol, PPG-1 glyceryl ether, PPG-9,2-pyrrolidone-5-carboxylic acid and its salts such as glyceryl pea, saccharide isomerate, SEACARE (available from Secma), sericin, silk amino acids, sodium acetylhyaluronate, sodium hyaluronate, sodium poly-aspartate, sodium polyglutamate, sorbeth 20, sorbeth 6, sugar and sugar alcools and derivatives thereof such as glucose, mannose and polyglycerol sorbitol, trehalose, triglycerol, trimethyolpropane, tris (hydroxymethyl) amino methane salts, and yeast extract, and mixtures thereof.

More preferably, the humectants for use herein are polyhydric alcohols selected from the group consisting of glycerin, diglycerin, glycerol, erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA and derivatives and mixtures thereof.

Other preferred embodiments may include the following humectants: aloe vera, cocoa butter, Vitamin E, honey, or a combination thereof.

In some other embodiments, the method of treatment comprises orally administering CAPE to a person in need thereof.

### Acne

The present inventors have realized that compositions comprising CAPE can lead to an improvement in acne symptoms in humans.

Specifically, the present disclosure provides a method of treating acne vulgaris or any one of the topical skin disorders that may include at least one of acne vulgaris, comedonic or polymorphic acne, nodulocystic acne, acne conglobata, senile acne or secondary acne, acne lesions, cystic acne, acne atrophica, bromide acne, chlorine acne, acne cosmetica, acne detergicans, epidemic acne, acne estivalis, acne fulminans, halogen acne, acne indurata, iodide acne, acne keloid, acne mechanica, acne papulosa, pomade acne, premenstrual acne, acne pustulosa, hidradenitis suppurativa, keratosis pilaris, acne rosacea, acne scorbutica, acne scrofulosorum, acne urticata, acne varioliformis, acne venenata, propionic acne, acne excoriee, pseudofolliculitis barbae, gram negative acne, steroid acne, pregnancy related acne, folliculitis, odulocystic acne, rosacea, common acne, seborrheic dermatitis, keratosis follicularis, perioral dermatitis, transient acantholytic dermatitis, dermatosis, focal acantholytic dyskeratosis, acne necrotica milliaris, seborrhea, comedones, papules, pustules, nodules, and pimples. All of these disorders are inclusive of the term acne as used herein.

In some other embodiments, the composition may consist essentially of CAPE. Specifically, the composition may exclude some unnecessary or extraneous components of bee propolis. For example, in some embodiments, the composition may not contain antepilin C, quercetin, chloredan diterpene, P-coumaric acid, cinnamic acid, or combinations thereof.

In other embodiments, the composition comprising CAPE may further comprise at least one other agent active toward acne. For example, some compositions may include salicylic acid and other salicylates, azelaic acid, Dapsone, benzoyl peroxide, zinc salts including zinc pyrithione and zinc sulfate, resorcinol, Vitamin A, Vitamin E, Vitamin B5, polyhydroxy acids (PHAs), alpha hydroxy acids (AHAs), beta hydroxy acids (BHAs), niacinamide, centella asiatica, cortisone, cortisone injection, hydrocortisone, hyaluronic acid, oligopeptides, hydrocolloid formulations, tea tree oil, retinol, and combinations thereof.

In some embodiments the composition may also include a keratolytic agent, which functions to soften and thin the epidermis to prevent clogged pores and release trapped sebum. For example, the composition may include a sulfur spot treatment, sodium sulfacetamide, or other sulfur-containing active agent, which, optionally may further be combined with resorcinol for additional therapeutic effect. Other keratolytic agents which may be present in some embodiments include urea, lactic acid, and allantoin.

In some embodiments, active anti-acne agents may comprise at least one prescription anti-acne drug. Non-limiting examples of such drugs may include isotretinoin (Accutane™), tretinoin (e.g., Avita™), adapalene (Differin™), tazarotene (e.g. Tazorac™), spironolactone, combination birth control, and combinations thereof.

In some embodiments, the composition comprising CAPE may further comprise an antibiotic active toward treating acne. Non-limiting examples of such antibiotics that may be administered in conjunction with CAPE include erythromycin, clindamycin, minocycline, and doxycycline. Moreover, the composition including both CAPE and an antibiotic may optionally further comprise benzoyl peroxide.

As one example of a composition including an antimicrobial agent, linoleic acid (octadeca-9,12 dienoic acid) has been reported in the art to exhibit antimicrobial effects against C. acnes and Propionibacterium strains, and linoleic acid may be utilized in certain embodiments to provide increased beneficial antibacterial properties. Hydrosylates of pure linoleic acid provide a cost benefit in the manufacturing of formulations as compared with pure linoleic acid, and may also form an active agent in some embodiments.

In other embodiments, the composition comprising CAPE may be administered to a subject who was previously undergoing a treatment regimen to treat acne. The previous treatment regimen is not particularly limited, but may include isotretinoin, salicylic acid, benzoyl peroxide, zinc salts including zinc pyrithione and zinc sulfate, resorcinol, Vitamin A, Vitamin E, and combinations thereof. The previous treatment may be a prescription medication or it may be an over-the-counter regimen, such as two-phase or combination salicylic acid treatment.

In addition to pain and discomfort, and a visually unaesthetic appearance, the presence of acne pimples may lead to the formation of scar tissue, which may itself be visually disfiguring for the patient for a much longer timeperiod than the experience of the acne itself. Therefore, some embodiments of the composition comprising CAPE may further comprise at least one anti-scarring product to eliminate or reduce the scarring caused by acne. The precise anti-scarring agent(s) is not particularly limited, and without limitation, may include glycolic acid, kojic acid soap, niacinamide, silicone sheeting, silicone gel, mediderm gel, Vitamin E oil, Derma E scar gel, hydrocolloid formulation, phenols, catechins, epigallocatechin gallate, rosehip oil, allicin, allantoin, onion extract, arbutin glycoside, hyaluronate, thyme oil, and combinations thereof.

In addition to optional other agents that are active for treatment of acne, some embodiments may include applying a composition comprising CAPE in conjunction with at least one acne treatment product. Without limitation, such further acne treatment products may include Cerave™ BHA face cleaner, Cerave™ moisturizer, Cetaphil™ gentle foaming cleanser at night, Peach and Lily™ salycitic acid toner, pimple patches, Zitsticka™ Deep Zit micro dart patches, Neutrogena™ light therapy acne spot treatment, TreeActiv™ Cystic Acne Spot, and combinations thereof. As used herein, the term “cleanser or wash” refers to a skin care product that is used to remove make-up, dead skin cells, oil, dirt, and other types of pollutants from the skin of the body and face. This helps to unclog pores and prevent and treat acne.

In some other embodiments, the method of treating acne comprises injecting a composition comprising CAPE to a subject in need thereof in a therapeutically effective amount and/or comprising from about 0.1 mg/kg to about 10 mg/kg. Without limitation, and solely by way of example, the injecting may be performed locally, subcutaneously, intraperitoneally, or intravascularly. Moreover, the time period of the injection is not particularly limited, such that the injection may be administered monthly, weekly, daily, or up to hourly for a set period of time.

It is also the case that non-human mammals, such as cats and dogs, mice, or any other mammals, occasionally suffer from acne. For example, cats and dogs are examples of mammals that are predisposed to acne. In dogs, the disease usually manifests in the facial region, especially in the lips and the skin around the muzzle. Unlike humans, cats and dogs often get acne in middle age rather than in their adolescent years. Therefore, all of the embodiments discussed herein may be applied to humans or, equivalently, to non-human mammals.

### Human Clinical Surveys

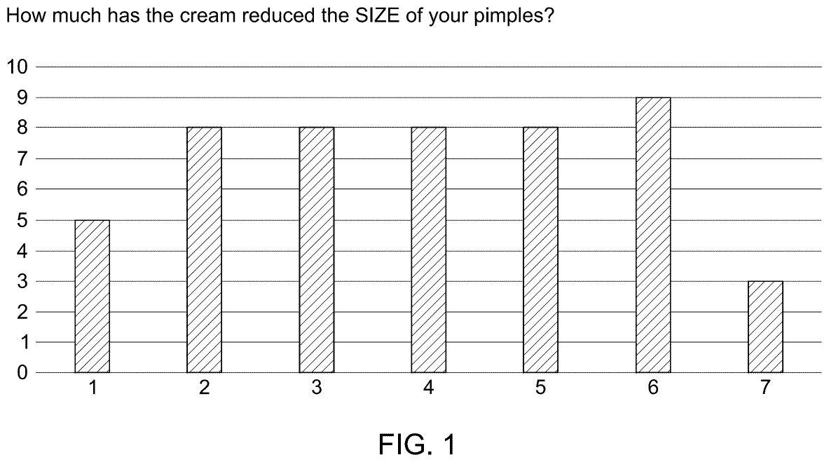

Seven individual participants who were experiencing acne pimples were given an acne spot cream comprising 10% CAPE. The participants were directed to apply it twice per day. The participants reported a score indicating how much the spot cream reduced the size of the pimples, wherein a value of 0 indicated no reduction and a value of 10 indicated maximum reduction. The results are shown in FIG. 1. As can be seen, all of the participants reported a reduction in pimple size. Moreover, five out of the seven participants reported a pimple size reduction of 8 or greater.

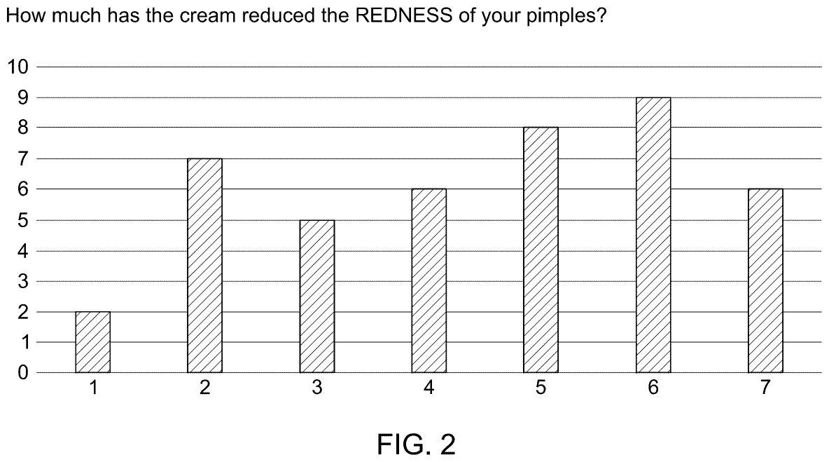

Similarly, the participants were asked to evaluate how much the cream had reduced the redness of their pimples, on the same scale as for pimple size. The results are shown in FIG. 2, where it can be seen that all seven participants reported a reduction in redness of their pimples. Moreover, three out of the seven participants reported a reduction score of 7 or greater.

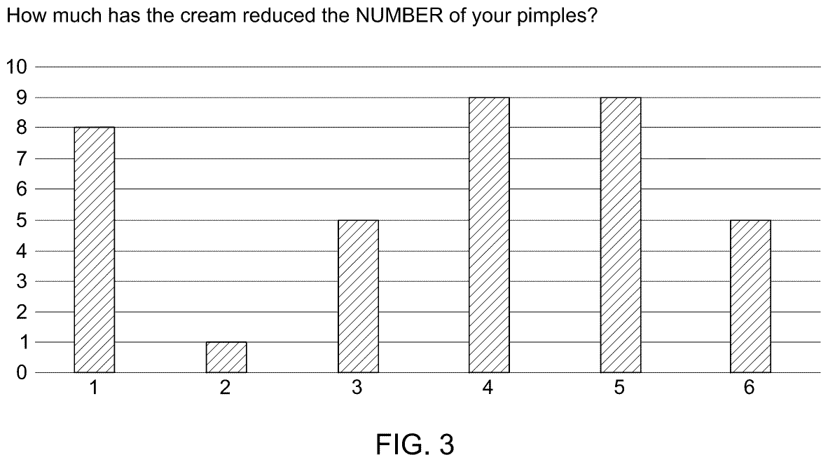

FIG. 3 shows the results of a study where six participants were asked to report how much the acne spot cream comprising CAPE reduced the number of their pimples. All six participants reported a reduction of the number of pimples. Three of the six participants reported a reduction score of 8 or greater.

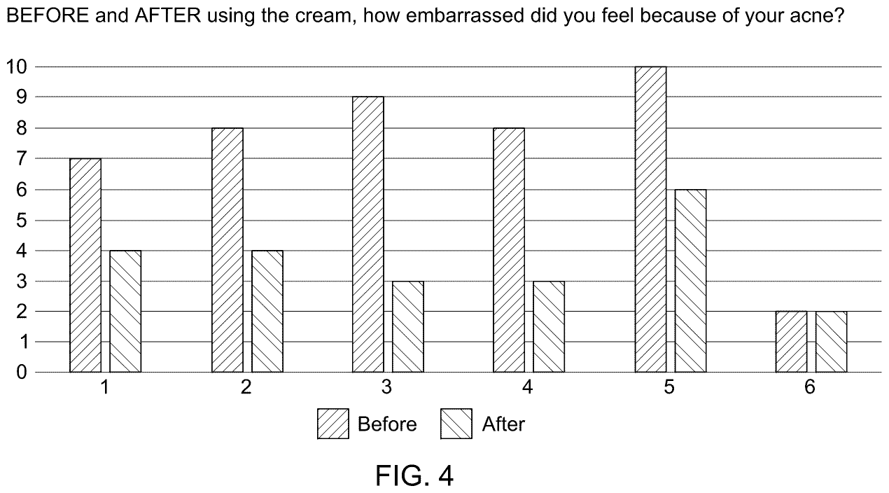

FIG. 4 shows the results of a study where six participants were asked to report a level of embarrassment before and after using the acne spot cream comprising CAPE. Five out of six participants reported a reduction in embarrassment as a result of using the spot cream.

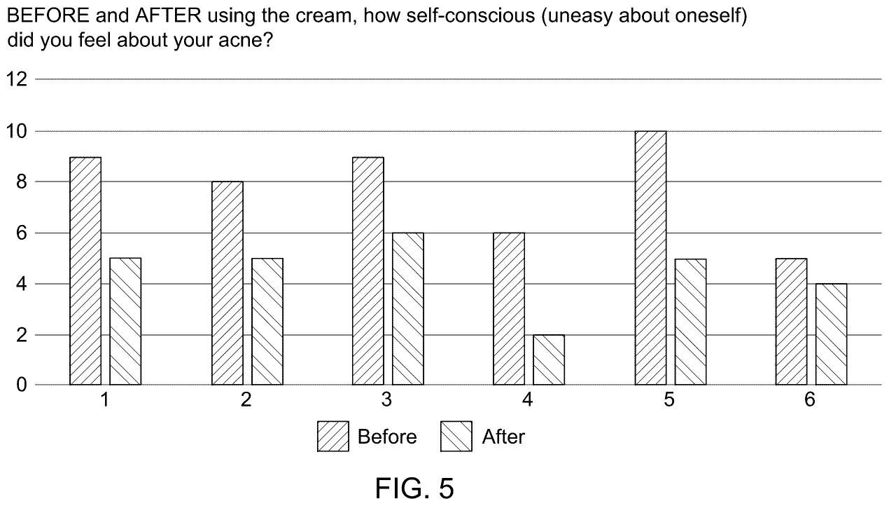

FIG. 5 shows the results of a study where six participants were asked to report a level of self-consciousness before and after using the acne spot cream comprising CAPE, where self-consciousness was defined as feeling uneasy about oneself. All six of the participants reported a reduction in self-consciousness as a result of using the spot cream.

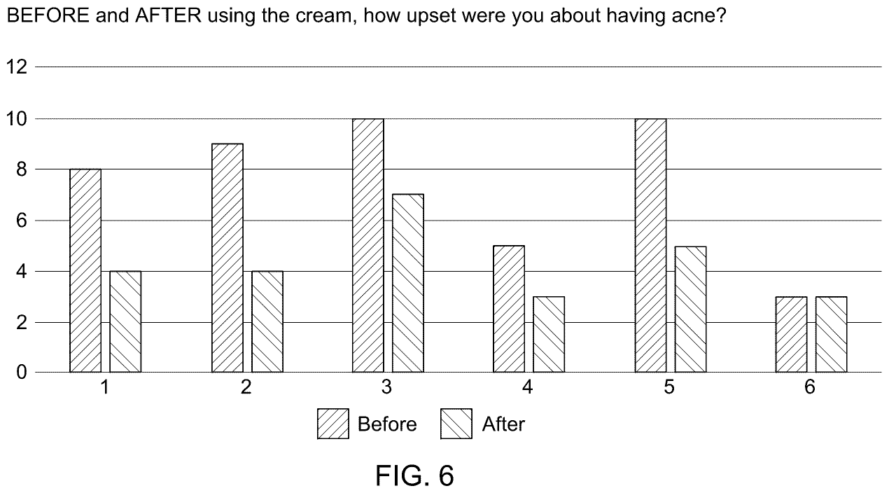

FIG. 6 shows the results of a study where six participants were asked to report how upset they were before and after using the acne spot cream comprising CAPE. Five of the six participants reported a decrease in how upset they were as a result of using the spot cream.

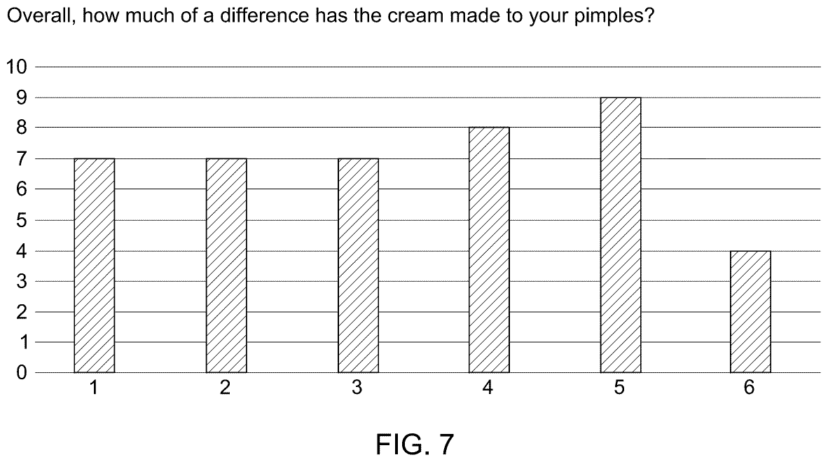

FIG. 7 shows the results of a study where six participants were asked to report how much, overall, a difference using the spot cream made to their pimples, where a level of 0 meant no difference and a level of 10 meant maximum difference. All six participants reported a difference in their pimples as a result of using the spot cream, with five out of six reporting a difference of 7 or greater.

All of the participants surveyed, when asked when they experienced any side effects from using the spot cream comprising CAPE, reported no side effects.

Finally, five out of the six participants surveyed, when asked if they would recommend the acne spot cream product comprising CAPE to family and friends, reported that they would recommend the product.

### UV or Radiation Induced Cutaneous Injury

The present inventors have realized that compositions comprising CAPE may lead to a reduction of symptoms of UV or radiation induced cutaneous injury, including sunburn.

In some embodiments, a method of treating UV or radiation induced cutaneous injury may include topically applying a composition comprising CAPE to a subject suffering from UV (e.g., sunburn) or other wavelength radiation induced cutaneous injury in a therapeutically effective amount. In the composition, the percentage of CAPE may be from about 1% to about 50% by weight.

The radiation induced damage may also occur to the oral or gastrointestinal mucosa of a subject in need thereof, such as a patient experiencing radiation therapy including chemotherapy. Such a condition is commonly known as mucositis. The present inventors have realized that compositions comprising CAPE may lead to a reduction of symptoms of mucositis, possibly by an anti-inflammatory method of action.

Therefore, some other embodiments may include a composition comprising CAPE that is applied via a “swish and spit” method. In a swish and spit method, a composition comprising CAPE is flushed inside a patient's mouth and swished around the mouth in order for the composition to adequately wash over the sites of mucositis.

### Scars

The present inventors have realized that compositions comprising CAPE may prevent and/or reduce the visual appearance of scars.

In some embodiments, a method of preventing and/or reducing the appearance of scars comprises topically applying a composition comprising CAPE to a subject in need thereof in a therapeutically effective amount. This therapeutically effective amount may comprise from about 1% to about 50% CAPE, by weight.

In some other embodiments, the composition comprising CAPE may be administered in conjunction with a scar cream product. There are numerous scar cream products known to the person skilled in the art. Some examples include Mederma Advanced Scar Gel™, SkinCeuticals Phyto+Botanical Gel™, Cica-Care Gel Sheet™, Cimeosil Scar and Laser Gel™, ScarAway Silicone Scar Gel™, Tosowoong Green Tea Natural Pure Essence™, Honeydew Scar Cream™, Mederma for Kids™, Derma E Scar Gel™, Rejuran Advanced Scar Cream™, Cera Ve Retinol Serum™, Musely the Spot Cream™, Differin Resurfacing Scar Gel™, SkinMedica Scar Recovery Gel™, and DermalMD Scar Treatment™.

### Mucositis

The present inventors have realized, through extensive studies, that compositions comprising CAPE may have beneficial effects in reducing the symptoms of mucositis.

In some embodiments, a composition comprising CAPE is administered to a patient experiencing mucositis. The composition may be administered in a dosage that is therapeutically effective, such dosage being readily discernable to the skilled person or, without limitation, comprising from about 1% to about 50% CAPE by weight.

In some preferred embodiments, the subject in need thereof is also undergoing cryotherapy, which may include ice chip therapy, to treat their mucositis. Such cryo or ice chip therapy may be prophylactic in nature. The use of ice chips or ice-cold water has been suggested to prevent or reduce the oncoming symptoms of oral mucositis. In some embodiments, the patient may hold ice or hold ice-cold water in their mouths prior to, during, and after rapid infusions of chemotherapy, or other agents constituting potentially mucotoxic agents. Cryotherapy is based on the theory that vasoconstriction caused by cold temperatures decreases the exposure of the oral cavity mucous membranes to mucotoxic agents. In some embodiments, the subject may be underoing infusion of fluoroacil, or 5-fluorouracil. In other preferred embodiments, the patient may be receiving melphalan.

In other embodiments of the present application, the liquid composition comprising CAPE may be administered with a mucoadhesive hydrogel rinse and/or a calcium phosphate rinse to treat mucositis. Non-limiting examples of mucoadhesive hydrogel rinses include MuGard (AMAG Pharmaceuticals, Waltham, MA). Non-limiting examples of calcium phosphate rinses efficacious toward mucositis include Caphosol (EUSA Pharma, Boston, MA). Both MuGard and Caphosol are approved by the United States Food and Drug Administration for the prevention of mucositis. Multiple studies have demonstrated a benefit in preventing mucositis in the oncology population.

In some other preferred embodiments, the liquid composition comprising CAPE is administered to a subject undergoing head and neck cancer radiotherapy. Radiotherapy targeting head and neck cancer has a significantly increased risk of affecting a subject's oral mucosa, despite the fact that usually significant efforts are made to avoid mucosal exposure, such as by using lead shielding to the extent appropriate.

In other preferred embodiments, the liquid composition comprising CAPE is administered before chemotherapy or radiotherapy to treat cancer in order to prevent or decrease the risk or severity of ensuing mucositis. Such administering may occur directly before the cancer therapy.

In other embodiments, the liquid composition is administered in conjunction with a a nonsteroidal anti-inflammatory drug, particularly a benzydamine hydrochloride mouthwash. These drugs can potentially inhibit the production of proinflammatory cytokines such as tumor necrosis factor-a and interleukin-13.

Additional agents of the anti-inflammatory drug category which may be present in preferred embodiments include diphenhydramine, prostaglandin E2, immunoglobulins, corticosteroids, indomethacin, azelastine, mesalazine, aspirin, orgotein, flurbiprofen, histamine, colchicine, and Placentrex.

Some preferred embodiments feature administering a liquid composition comprising CAPE along with an opiate mouthwash, where the opiate mouthwash may comprise morphine, fentanyl, or codeine. Systemic opiate agents, for example a 2% morphine swish and spit mouthwash, have been suggested to achieve pain control with limited systemic absorption and may reduce the severity of mucositis.

Some embodiments may further include normal saline or sodium bicarbonate solutions, which have been suggested to provide relief of mild to moderate mucositis pain.

Such salt-and-soda mouthwashes are also generally regarded as safe, inexpensive, and effective in alleviating mucositis symptoms. These mouthwashes can be performed as frequently as every 4 hours.

Other preferred embodiments further comprise a liquid composition comprising CAPE in conjunction with an anesthetic, an antihistamine, an antibiotic, or a steroid. Such combination of active ingredients in a swish-and-spit formulation are generally called a “magic mouthwash.” For example, lidocaine may be highly effective in conjunction with CAPE to provide additional pain relief. In some preferred embodiments, the magic mouthwash may comprise a steroid, with especially preferred embodiments comprising dexamethasone. Dexamethasone mouthwash has been shown to be a safe, effective, and inexpensive therapy for stomatitis in patients suffering from metastatic breast cancer.

Obviously, numerous modifications and variations of the present invention are possible in light of the above teachings. It is therefore to be understood that within the scope of the appended claims, the invention may be practiced otherwise than as specifically described herein.

1. A method of treating acne, comprising:
topically applying a composition comprising Caffeic Acid Phenethyl Ester (CAPE) to a subject in need thereof in a therapeutically effective amount and/or comprising from about 1% to about 50% CAPE by weight.

2. A method of treating UV or other radiation induced injury, comprising:
topically applying a composition comprising Caffeic Acid Phenethyl Ester (CAPE) to a subject in need thereof in a therapeutically effective amount and/or comprising from about 1% to about 50% CAPE by weight.

3. A method of preventing and/or reducing the appearance of scars, comprising:
topically applying a therapeutically effective amount of a composition comprising Caffeic Acid Phenethyl Ester (CAPE) to a subject.

4. (canceled)

5. The method of claim 3, wherein the composition further comprises a pharmaceutically acceptable base.

6. The method of claim 5, wherein the pharmaceutically acceptable base is a water and oil emulsion, dispersion, suspension, colloid, liposome, microemulsion, nanoemulsion, clear solution, or anhydrous composition.

7. The method of claim 5, wherein the pharmaceutically acceptable base comprises at least one of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, or skin surface implant.

8. The method of claim 5, wherein the pharmaceutically acceptable base further comprises at least one of
at least one skin-beneficial ingredient;
ii) at least one solubilizing-penetration enhancing agent:
if) an antibiotic:
iv) an anti-inflammatory agent:
v) at least one anti-scarring agent;
vi) at least one analgesic agent:
vii) at least one emollient:
viii) an anti-acne agent; and/or
ix) a prescription acne drug.

9. The method of claim 8, wherein the composition comprises at least one solubilizing-penetration enhancing agent, which is an alcohol, a hydroxy acid ester or a glycol.

10. (canceled)

11. The method of claim 8, wherein the composition comprises at least one antibiotic, which is erythromycin, clindamycin, minocycline, or doxycycline, optionally wherein the antibiotic is active against Cutibacterium acnes, Staphylococcus epidermidis, or Staphylococcus aureus

12-13. (canceled)

14. The method of claim 8, wherein the composition comprises at least one anti-inflammatory agent, which is clindamycin, erythromycin, dapsone, aloe vera or tea tree oil.

15-17. (canceled)

18. The method of claim 8, wherein the composition comprises at least one analgesic agent, which is aloe vera, lidocaine, capsaicin, menthol, or a nonsteroidal anti-inflammatory drug (NSAID).

19-27. (canceled)

28. The method of claim 3, wherein the composition does not contain antepilin C, quercetin, chloredan diterpene, P-coumaric acid and/or cinnamic acid.

29. The method of claim 3, wherein the composition further comprises at least one anti-acne agent, which is
salicylic acid or derivative thereof, ester of a salicylic acid or derivative thereof, azelaic acid, Dapsone, benzoyl peroxide, zinc salts including zinc pyrithione and zinc sulfate, resorcinol, Vitamin A, Vitamin E, Vitamin B5, polyhydroxy acids (PHAs), alpha hydroxy acids (AHAs), beta hydroxy acids (BHAs), niacinamide, centella asiatica, cortisone, cortisone injection, hydrocortisone, hyaluronic acid, oligopeptides, hydrocolloid formulations, tea tree oil or retinol.

30-54. (canceled)

55. The method of claim 3, wherein the composition comprises about 1% to about 50% of CAPE by weight.

56. The method of claim 3, wherein the composition is liquid, aqueous or semi-solid.

57. The method of claim 8, wherein the composition comprises at least one skin-beneficial ingredient, which is a skin cleanser, surfactant, skin and hair conditioning agent, vitamin, hormone, mineral, plant extract, anti-inflammatory agent, concentrate of plant extract, emollient, moisturizer, skin protectant, humectant, silicone, skin soothing ingredient, analgesic, skin penetration enhancer, solubilizer, moisturizer, anesthetic, colorant, perfume, preservative, seed, broken seed nut shell, silica, clay, bead, luffa particle, polyethylene ball, mica, pH adjuster, or processing aid.

58. The method of claim 8, wherein the composition comprises at least one anti-scarring agent, which is glycolic acid, kojic acid soap, niacinamide, silicone sheeting, silicone gel, mediderm gel, Vitamin E oil, Derma E scar gel, hydrocolloid formulation, phenols, catechins, epigallocatechin gallate, rosehip oil, allicin, allantoin, onion extract, arbutin glycoside, hyaluronate or thyme oil.

59. The method of claim 8, wherein the composition comprises at least one emollient, which is a fatty ester, a fatty alcohol, petrolatum, or dimethicone.

60. The method of claim 8, wherein the composition comprises at least one prescription anti-acne drug, which is isotretinoin, tretinoin, adapalene, tazarotene, spironolactone, or combination birth control.

61. The method of claim 3, wherein the applying topically is conducted monthly, weekly, daily, or up to six times per day
